(19) United States
(12) Patent Application Publication
(10) Pub. No.: US 2014/0343116 A1
(43) Pub. Date: Nov. 20, 2014

(54) CYTOCHROME P450 2C9 INHIBITORS

(71) Applicant: NATIONAL DEFENSE MEDICAL CENTER, TAIPEI CITY (TW)

(72) Inventors: Oliver Yoa-Pu HU, Taipei (TW); Hong-Jaan Wang, Pingtung County (TW); Cheng-Huei Hsiong, Taipei (TW); Li-Heng Pao, Taipei (TW)

(21) Appl. No.: 14/447,587

(22) Filed: Jul. 30, 2014

Related U.S. Application Data
(62) Division of application No. 12/628,194, filed on Nov. 30, 2009, now abandoned.

(51) Int. Cl. A61K 31/405

(52) U.S. Cl. 514/419

(57) ABSTRACT

This invention is to provide multiple specific inhibitors of cytochrome P450 isozyme CYP2C9. These inhibitors can be derived from any combinations with the following compounds including: Tamarixetin, Formononetin, isoliquritigenin, Phloretin, luteolin, Quercitrin, quercetin, myricetin, Wongonin, Puerarin, Genistein, Nordihydroguaiaretic acid, Narigenin, Capillarisin, Chrysin, Fisefin, eriodictyol, 6-Gingerol, Isorhamneti, isoquercitrin, Morin, (+)-Taxifolin, isovitexin, 3-Phenylpropyl Acetate, Oleanolic acid, ursolic acid, β-Myrcene, cinnamic acid, Luteolin-7-Glucoside, Liquiritin, (+)Limonene, Homoorientin, Swertiamarin, Embelin, Daidzein, Poncirin, (−)-Epicatechin, ergosterol. These natural products can be used to enhance the bioavailability of therapeutic agents (drugs).

### RELATED APPLICATIONS

This application is divisional application of U.S. patent application Ser. No. 12/628,194, filed on Nov. 30, 2009, which is a continuous application of U.S. patent application Ser. No. 10/948,206, filed on Sep. 24, 2004.

### BACKGROUND OF THE INVENTION

This invention is to provide inhibitors of cytochrome P450, especially inhibitors that are specific for the isoform CYP2C9.

Cytochrome P450 (P450) is the most important oxidative enzymes for the metabolism of drugs and xenobiotics. P450 is classified as families and subfamilies, and is widely distributed in the liver, intestines and other tissues (Krishna D. and Klotz U., Extrahepatic metabolism of drugs in humans. Clinical Pharmacokinetics. 26:144-160, 1994). Cytochrome P450 enzymes catalyze the phase 1 reaction of drug metabolism, to generate metabolites for excretion. The classification of CYP450 is based on homology of the amino acid sequence (Slaughter R. L. and Edward D. J., Recent advances: the cytochrome P450 enzymes. The Annals of Pharmacotherapy. 29:619-624, 1995). In mammals, there is over 55% homology of the amino acid sequence of CYP450 subfamilies. The differences in amino acid sequence constitute the basis for a classification of the superfamily of cytochrome P450 enzymes into families, subfamilies and isozymes. The isozymes with similar numerical numbers (for example CYP2C9 and CYP2C11, CYP1A1 and CYP1A2) usually have high amino acid homology, and their respective genes usually locate in proximate positions on the chromosome map. For instance, CYP2C9 and CYP2C10 have only two amino acid differences; the amino acid sequence homology of CYP3A3 and CYP3A4 is 97.5%. Therefore, the nomenclature of cytochrome P450 is across all living systems and species, including animals, plants and microorganisms. Cytochrome contains an iron cation and is a membrane bound enzyme. The hemoprotein structure (heme-group, prosthetic group) and function of P450 are very similar to those of hemoglobin, it can carrier out electron transfer and energy transfer. Cytochrome P450, when binds to carbon monoxide (CO), displays a maximum absorbance (peak) at 450 nm in the visible spectra, and is therefore called P450 (Omura T. and Sato R., The carbon monoxide-binding pigment of liver microsomes. The Journal of Biological Chemistry. 239:2370-2378, 1964).

### CYP450 Tissue Distribution:

Regarding tissue distribution of CYP450, there is a great similarity between rats and humans. Human CYP450 isozymes are widely distributed among tissues and organs (Zhang Q. Y., Dunbar D., Ostrowska A., Zeisloft S., Yang J., and Kaminsky L. S., Characterization of human small intestinal cytochromes P-450. Drug Metabolism and Disposition. 27:804-809, 1999). With the exception of CYP1A1, most human CYP450 isozymes are located in the liver, but are expressed at different levels (Waziers I., Cugnenc P. H., Yang C. S., Leroux J. P. and Beaune P. H., Cytochrome P450 isoenzymes, expoxide hydrolase and glutathione transferases in rat and human hepatic and extrahepatic tissues. The Journal of Pharmacology and Experimental Therapeutics. 253:387-394, 1990). For example, CYP2C family constitutes about 18.2% of the total P450 in the liver. Human intestine also has high CYP3A4 contents, approximately 50% of that in the liver. The distribution in rats is similar to humans. With the exception of CYP2B1 and CYP1A1, the majority of the known rat CYP450 isozymes are primary located in the liver. From literatures, it's also known there are species differences in the tissue distribution and expression of CYP450 enzymes between rats and humans. However, from the enzymatic and functional perspectives, the rat P450 enzymes are considered representative of the human enzymes. Consequently, Sprague-Dawley rat liver microsomes are used as an enzyme source for investigating CYP2C.

Fifty-seven CYP450 isozymes have been identified from the human CYP genomics, and they have been classified into fourteen P450 subfamilies—CYP 1, 2, 3, 4, 5, 7, 8, 11, 17, 19, 21, 24, 27 and 51(Nelson D. R., Koymans L. and Kamataki T., P450 superfamily: update on new sequences, gene mapping, accession numbers and nomenclature. Pharmacogenetics. 6:1-42, 1996). CYP1, 2 and 3 are primary responsible for metabolism and detoxication of drugs and xenobiotics. The other 11 P450 subfamilies are responsible for the catabolism of endogenous compounds, such as hormones or steroids, etc.

### Genetic Polymorphism

Presently, four isoforms have been identified for human CYP2C subfamily. They are CYP2C8, CYP2C9, CYP2C18 and CYP2C19, and there are about 82% amino acid sequence homology among these four isoforms (Miners J. O. and Birkett D. J., Cytochrome P4502C9: an enzyme of major importance in human drug metabolism. British Journal of Clinical Pharmacology. 45:525-538, 1998). Despite the high homology, there are large differences in substrate specificity among these isoforms. It is also reported in 1980's that genetic polymorphism existed for CYP2C subfamily, as is observed for CYP2D6. Since then, many clinical studies have been performed to investigate the polymorphism of CYP2C. Results of these studies concluded that human populations can be categorized into two groups based on drug metabolism CYP450 activities: extensive metabolizers (EMs) and poor metabolizers (PMs). The ratios of this genetic polymorphism are different among different races. For example, approximately 2 to 4% of the Caucasians populations are PMs, while there are 20% in Asians. Consequently, drug-drug interactions mediated by substrate specific metabolic pathways can be a more significant issue in Asian population.

### Drug Metabolism

Following absorption and reaching systemic circulation, drug molecules undergo metabolism and elimination/excretion process. There are two major metabolic reactions—phase I reaction and phase II reactions, both leading to more hydrophilic metabolite(s). The formation of hydrophilic metabolites is to facilitate excretion from the body. Mixed function monooxygenase is the major enzyme responsible for phase I reaction. Cytochome P450 is a monooxygenase system, consisting of P450, P450 reductase, cytochrome b5. These proteins function together to catalyst the reduction/oxidation of drug molecules, the mechanism of these reactions is described in the sections follow. Phase II reactions are primary conjugation reactions, can be divided into six categories (Table 1). Glucronidation, sulfation and glutathione conjugation are the most commonly observed phase II reactions.

TABLE 1
Drug Phase I and Phase II reactions (Shargel L., and
Yu A. B. C., Hepatic elimination of drugs. Applied
Biopharmaceutics and Pharmacokinetics. 4th ed., Appleton
& Lange, Stamford, pp. 353-398, 1999)

| | Phase II reaction |
|---|---|
| Phase I reaction | (High energy intermedate) |
| Oxidation | Glucuronide conjugation (UDPGA) |
| Aromatic hydroxylation | |
| Aliphatic hydroxylation | Sulfate conjugation (PAPS) |
| N-, O-oxidation | |
| N-, O-dealkylation | Glutathion conjugation (GSH) |
| Deamination | |
| Reduction | Acetylation (Acetyl coenzyme A) |
| Azoreduction | |
| Nitroreduction | Methylation (SAM) |
| Alcohol dehydrogenase | |
| Hydrolysis | |
| Ester hydrolysis | |
| Amide hydrolysis | |

UDPGA = uridine diphosphoglucuronic acid,
PAPS = 3′-phosphoadenosine 5′-phosphosulfate,
GSH = glutathione,
SAM = S-adenoylmethionine

The four CYP2C isozymes have different substrate specificity, however, metabolism of most drug molecules is carried out by CYP2C9 and CYP2C19. The relative activity of CYP2C9 and CYP2C19 in human liver is about 3:1 (Venkatakrishnan K., von Moltke L. L., Greenblatt D. J., Relative quantities of catalytically active CYP 2C9 and 2C19 in human liver microsomes: application of the relative activity factor approach. Journal of Pharmaceutical Sciences. 87:845-53, 1998). One of a commonly used proton pump inhibitor, Omeprazole, is a specific substrate for CYP2C19. CYP2C9 exhibits broader substrate selectivity and metabolizes different classes of drug, including non-steroid anti-inflammatory drug (NSAID's), blood triglyceride lowering agents, anti-coagulants. Representative examples are listed in Table 2. It should be noted that phenytoin and warfarin (on the lists) are clinical agents with narrow therapeutic window. For these agents, changes in oral absorption due to individual variability or other environmental factors can lead to severe side effects and undesired treatment outcome. One of the causes in individual variability is genetic polymorphism. The pattern of genetic polymorphism is different among races. For example, CYP2D6 is an enzyme responsible for the metabolism of hydrophobic anti-depressants. About 19% of the Caucasian population is CYP2D6 poor metabolizer (PMs), in contracts, the CYP2D6 PMs among oriental populations is less than 1%. Therefore, when a standard therapeutic dose of an anti-depressant is given to a PM patient, severe side effects are often observed because of the reduced metabolism rate in a PM. These side effects compromise the quality of life and further reduce patient compliance, and even accelerate the disease progression. Similarly, when a narrow therapeutic window drug is given to a PM patient, severe adverse effects can result due to reduced metabolism rate.

To address the issue of variability in drug bioavailability, one approach is to control drug absorption (for example, use of control released drug product). Another and a more direct approach is to control the rate of drug metabolism. When the rate of absorption and rate of metabolism reach a steady state, a maintenance dose can be deliver to achieve the desired drug level (systemic availability) that is required for drug efficacy. This approach will minimize the individual variability, avoid side effects. Furthermore, by searching/use of an effective P450 inhibitor, the drug metabolism rate can be regulated and drug first pass effects can be reduced. However, an effective P450 inhibitor has to process an acceptable safety profiles. For instance, natural products or Chinese herbal medicines can fulfill these safety requirements. One of most commonly observed examples for a natural product to alter (increase) the bioavailability of a drug is the effects of grape fruit juice on the pharmacokinetics of felodipine and other drug products (Edgar et al., Acute effects of drinking grapefruit juice on the pharmacokinetics and dynamics of felodipine—and its potential clinical relevance. European Journal of Clinical Pharmacology. 42:313-317, 1992; Lee et al., Grapefruit juice and its flavonoids inhibit 11 beta-hydroxysteroid dehydrogenase. Clinical Pharmacology and Therapeutics. 59:62-71, 1996; Kane et al., Drug-grapefruit juice interactions. Mayo Clinic Proceedings. 75(9):933-42, 2000).

TABLE 2
Substrates, Inhibitors and Inducers of CYP2C subfamilies (Rendic
S., Summary of information on human CYP enzymes: human P450
metabolism data. Drug Metabolism Reviews. 34: 83-449, 2002)

| Isoenzyme | Substrate | Inhibitor | Inducer |
|---|---|---|---|
| CYP2C9 | Tolbutamide | Fluconazole | Rifampin |
| | Diclofenac | Ketoconazole | Phenobarbital |
| | Warfarin | Metronidazole | Cabamazepine |
| | Phenytoin | Itraconazole | Ethanol |
| | Torsemide | Cimetidine | |
| | Fluvastatin | Sulphaphenazole | |
| | Losartan | Phenylbutazone | |
| | Celecoxib | | |
| | Meloxicam | | |
| | Isoniazide | | |
| | Valporic acid | | |
| | Ibuprofen | | |
| | Carvedilol | | |
| | Naproxan | | |
| | Ondansetron | | |
| CYP2C19 | Omeprazole | Fluoxetine | Rifampin |
| | Imipramine | Sertraline | Hexobarbital |
| | Diazepam | Ritonavir | |
| | Mephenytoin | | |
| | Clomipramine | | |
| | Propanolol | | |

### BRIEF SUMMARY OF THE INVENTION

This invention employ rat liver microsomes as an in vitro model and tolbutamide (Orinase®, a hypoglycemic agent) as a probe (marker) substrate (tolbutamide is 90% metabolized by CYP2C9) to measure the inhibition of CYP2C9. Test compounds are purified extracts from Chinese herbal medicines and natural products. The inhibitory effects towards the in vitro microsomal metabolism of tolbutamide are measured and CYP2C9 inhibitors are identified. These inhibitors can be used as in vivo CYP2C9 inhibitors leading to improve the bioavailability of other therapeutic agents.

First, this invention provides effective CYP2C9 inhibitor(s). These specific CYP2C9 inhibitors are derived from any combinations with the following compounds: Tamarixetin, Formononetin, isoliquritigenin, Phloretin, luteolin, Quercitrin, quercetin, myricetin, Wongonin, Puerarin, Genistein, Nordihydroguaiaretic acid, Narigenin, Capillarisin, Chrysin, Fisefin, eriodictyol, 6-Gingerol, Isorhamnetin, isoquercitrin, Morin, (+)-Taxifolin, isovitexin, 3-Phenylpropyl Acetate, Oleanolic acid, ursolic acid, β-Myrcene, cinnamic acid, Luteolin-7-Glucoside, Liquiritin, (+)Limonene, Homoorientin, Swertiamarin, Embelin, Daidzein, Poncirin, (−)-Epicatechin, ergosterol.

Secondly, this invention is to provide a pharmaceutical combination to improve the bioavailability of drug products extensively metabolized by CYP2C9. This pharmaceutical combination(s) contain the purified ingredient(s) from the essential and adjuvant components of Chinese medicines and pharmaceutical drug carrier. The purified ingredient(s) from the essential and adjuvant components of Chinese medicines act as CYP2C9 inhibitor(s), and are derived from the combination of the following: Tamarixetin, Formononetin, isoliquritigenin, Phloretin, luteolin, Quercitrin, quercetin, myricetin, Wongonin, Puerarin, Genistein, Nordihydroguaiaretic acid, Narigenin, Capillarisin, Chrysin, Fisefin, eriodictyol, 6-Gingerol, Isorhamnetin, isoquercitrin, Morin, (+)-Taxifolin, isovitexin, 3-Phenylpropyl Acetate, Oleanolic acid, ursolic acid, β-Myrcene, cinnamic acid, Luteolin-7-Glucoside, Liquiritin, (+)Limonene, Homoorientin, Swertiamarin, Embelin, Daidzein, Poncirin, (−)-Epicatechin, ergosterol). The pharmaceutically viable drug is one selected from the group consisting of tolbutamide, diclofenac, warfarin, phenytoin, torsemide, fluvastatin, losartan, celecoxib, meloxicam, isoniazide, valproic acid, ibuprofen, carvedilol, naproxen, and ondansetron.

The better inhibitor from the above lists is Tamarixetin.

A pharmaceutical combination contains tolbutamide and when used as a combination drug therapy, the purified ingredient(s) from the essential and adjuvant components of Chinese medicines can increase the bioavailability of tolbutamide.

A pharmaceutical combination contains fluvastatin and when used as a combination drug therapy, the purified ingredient(s) from the essential and adjuvant components of Chinese medicines can increase the bioavailability of fluvastatin.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

These features and advantages of the present invention will be fully understood and appreciated from the following detailed description of the accompanying Drawings.

### BRIEF DESCRIPTION OF THE DRAWINGS

FIG. 1 is the in vitro effects of Ketoconazole on 4′-hydroxylation of tolbutamide in liver microsomes.

FIG. 2 is the comparison of cytochrome P450 inhibitory activities of the top ten tested essential and adjuvant components of Chinese medicines at a testing concentration of 100 μM.

FIG. 3 is the comparison of cytochrome P450 inhibitory activities of the top ten tested essential and adjuvant components of Chinese medicines at a testing concentration of 10 μM.

FIG. 4 is the comparison of cytochrome P450 inhibitory activities of the top ten tested essential and adjuvant components of Chinese medicines at a testing concentration of μM.

FIG. 5 is the in vitro effects of tamarixetin on 4′-hydroxylation of tolbutamide in liver microsomes.

FIG. 6 is the blood concentration time profiles following oral administration of fluvastatin in Sprague-Dawley rats; n=5 for dosed group and n=7 for vehicle control group.

FIG. 7 is the in vitro effects of isoliquritigenin on 4′-hydroxylation of tolbutamide in liver microsomes.

FIG. 8 is the in vitro effects of Genistein on 4′-hydroxylation of tolbutamide in liver microsomes.

### DETAILED DESCRIPTION OF THE PREFERRED EMBODIMENTS

This invention focuses on the identification of CYP2C9 inhibitors. As reported in literature, inhibition patterns of tolbutamide metabolism in rat, rabbit, dog, micropig, monkey and man liver microsomes revealed a high degree of similarity between the dog and human sytems. However, from the enzyme kinetic aspects, the kinetic parameters (Vmax/Km) values for the rat and human systems are most comparable (Bogaards et al., Determining the best animal model for human cytochrome P450 activities: a comparison of mouse, rat, rabbit, dog, micropig, monkey and man. Xenobiotica. 30:1131-1152, 2000). When comparing the in vivo metabolism across species, it is reported that the biotransformation pathways of tolbutamide are similar in rat, rabbits and humans, and there is a species differences between dog and man (Dogterom P. & Rothuizen J., A species comparison of tolbutamide metabolism in precision-cut liver slices from rats and dogs. Drug Metabolism and Disposition. 21:705-709, 1993). Furthermore, the amino acid sequence of rat and human CYP2C9 and CYP2C11 reveals 73% homology (www.drnelson.utmem.edu/CytochromeP450/html), the biological functionality of these enzymes reveals 84% similarity (www.ncbi.nlm.nih.gov/BLAST/). On the basis of these findings, it is prudent to use rat as an in vivo and in vitro model to assess the inhibition potential of testing compounds against human liver CYP2C9.

This invention utilize the purified components from Chinese medicines to perform both in vitro inhibition and in vivo animal studies, the aims are to investigate their potential effects on the pharmacokinetics of drugs with low bioavailability, and to identify potential cytochrome P450 inhibitors from the essential and adjuvant components of Chinese medicines.

### Materials and Methods

The essential and adjuvant components Chinese medicines employed in this invention are purified chemical components from commonly used Chinese medicines. Their chemical structures can be classified into five (5) categories: flavones, flavanones, chalcones, isoflavones and coumarins.

1. Preparation of Liver Microsomes

This invention use rat as the experimental animal model, therefore, in vitro enzymes used for metabolism studies are also prepared from rat liver.

After sacrifice, the liver is removed from the rats and placed in 1.15% potassium chloride at 4° C. The tissue is thoroughly rinsed with cold 1.15% potassium chloride solution to remove any residual blood, blot and weighed. The rinsed tissue is then homogenized in a high speed tissue homogenizer until a complete homogenate (no residual tissue chunks) is obtained (homogenizing tubes are pre-chilled on ice).

The homogenate is transferred to centrifuge tubes and centrifuged at 12,500×g for 20 minutes to remove cellular debris, nuclei, mitochondria and lysosomes. The supernatant fractions are harvested and placed into ultracentrifuges tubes (5 to 6 mL per tube). The tubes are then centrifuged in an ultracentrifuge at 100,000×g for 2 hours. The resulting supernatant (cytosol fractions) is discarded and the residual supernatant inside the centrifuge tubes are rinsed and removed cold 1.15% potassium chloride. The pellets (microsomes) are then harvested and resuspended in 0.1 M pH 7.4 phosphate buffer (one mL/g liver tissue).

The final liver microsomal preparation had a protein concentration of approximately 25 mg/mL, and is stored in a −80° C. freezer. Under this storage conditions, the enzymatic activities is unchanged for at least 8 weeks, and is suitable for drug metabolism studies. To avoid any experimental artifacts, the liver microsomes preparation should be used within the recommended storage stability timeframe. The microsomes preparation is summarized in the following steps:
(1) Animal sacrifice
(2) Removal of liver tissue
(3) Rinse liver tissue and record weigh of the tissue
(4) Cut the tissue into small pieces and mix with 1.15% KCL (1 mL/g tissue)
(5) Completely homogenize the tissue
(6) Place in high speed centrifuge tubes (12 to 15 mL per tube)
(7) Centrifuge at −4° C., 12,500×g, 20 minutes
(8) Place the supernatant into ultracentrifuge tubes
(9) Ultracentrifuge at −4° C., 100,000×g, 2 hours
(10) Discard supernatant, rinse the inside of centrifuge tubes with 1.15% KCL
(11) Remove the pellets from the centrifuge tubes
(12) Add pH 7.4 phosphate buffer, one mL per g of original tissue
(13) After respansion in phosphate buffer, dispense into micocentrifuge tubes (1 mL/tube)
(14) Store frozen at approximately −80° C. (−80° C. freezer)

2. In Vitro CYP2C9 Activity Assay for Screening of the Essential and Adjuvant Components of Chinese Medicines

After preparation and determination of microsomal protein concentrations, CYP2C9 activity assay are performed using the microsomes preparation, as a screen CYP2C9 inhibitors. Prior to screening, in vitro assay conditions are established based on enzyme kinetic principals and relevant kinetic parameters.

Tolbutamide is a specific substrate for human CYP2C9. CYP2C9 catalyzes the conversion of tolbutamide to a hydrophilic metabolite, 4′-hydroxytolbutamide. This metabolic reaction has been shown to be CYP2C9 specific and does not involved other P450 isozymes. Thereby, it is considered as a reliable measurement for CYP2C9 activity. The initial substrate concentration used is 1 mM, under a enzyme saturating condition (Tang et al., Effect of albumin on phenytoin and tolbutamide metabolism in human liver microsomes: an impact more than protein binding. Drug Metabolism and Disposition. 30:648-654, 2002).

The enzymatic assay conditions in microsomes are as following (total volume=1 mL):
(1) 0.1M phosphate buffer, pH 7.4
(2) 0.5 mg microsomal protein
(3) 5 mM magnesium chloride
(4) 10 mM glucose 6-phosphate
(5) 2 IU G6P dehydrogenase
(6) 1 mM β-nicotinamide adenine dinucleotide phosphate
(7) 1 mM tolbutamide
(8) 1% methanol

The activity assay mixture is placed on ice to maintain a 4° C. After the addition of the cofactor cocktails, it is pre-incubated in a 37° C. water bath for 1 minute. Reaction is initiated by the addition of the substrate and is terminated by 1N hydrochloric acid (0.1 mL). The metabolic reaction product is extracted using 2 mL methylene chloride. After separation by centrifugation, the organic fraction is concentrated to dryness, constituted in appropriate solvent and then analyzed for the metabolite (product) concentration.

The assay conditions are established as such product formation is linear with respect to incubation time and protein concentrations. In additions, initial substrate concentrations are selected based on the values of kinetic parameters, Km and Vmax.

The reaction product (metabolite) is analyzed using high performance liquid chromatotograhy (Shimadzu Model LC-10AD), UV detector (Shimadsu SPD-10A) at wavelength 230 nM (Miners et. al 1988). The LC conditions are, C-18 column (150×4.6 mm), mobile phase (10 mM acetate, pH 4.4/acetonitrile 25:75 v/v), flow rate 1.3 mL/min, ambient temperature. The retention time for the metabolite, internal standard and the substrate is 4.6, 14.2 and 26.5 minutes, respectively.

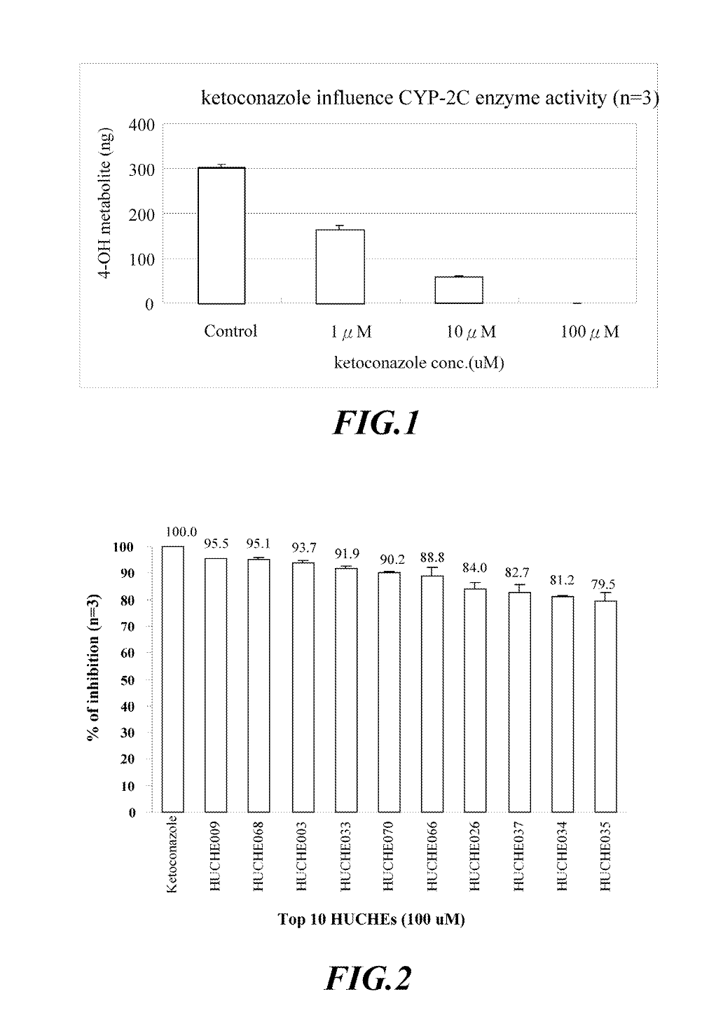

Ketoconazole is used as the positive control and are tested under different concentrations to demonstrate concentration dependency (Results shown in FIG. 1). At 100 μM concentrations, ketoconazole completely abolished the activity of the microsomes, exhibiting 100% inhibition. A 80.1 and 45.9% inhibition is observed at 10 and 1 μM, respectively.

On the basis of inhibitory activity observed for the positive control, screening of inhibitors from essential and adjuvant components of Chinese medicines is carried out at high, mid and low concentrations. However, the aqueous solubility of the essential and adjuvant components of Chinese medicines is relatively poor, and organic co-solvents (such as methanol, ethanol, acetonitrile) are usually used under the assay conditions. Consequently, solvents effects (vehicle control) on the enzymatic activities are assessed to eliminate experimental artifacts due to organic co-solvents.

3. In Vivo Study in Rodents

Potential inhibitors identified from the in vitro screen (using rat liver microsomes as an enzyme source and triglyceride lowering drug tolbutamide as a probe substrate) are subject to further in vivo evaluation in small animals. The test system used is the Sprague-Dawley rat. However, since the oral bioavailability of tolbutamide in rats is 90%, therefore, it is not an appropriate model compound for in vivo assessment. Blood cholesterol lowering agent, fluvastatin is used as a model compound. Fluvastatin is a synthetic HMG-CoA reductase inhibitor, its oral bioavailability is about 25 to 30% and it is predominantly metabolized by CYP29. Absorption of fluvastatin sodium following oral administration is about 90%, therefore, the low bioavailability of 25 to 30% is due to high first pass effects. Fluvastatin is metabolized in liver, forming four major metabolites (Scripture et. al 2001). Liver CYP2C9 is responsible for approximately 80% of fluvastatin metabolism, and other isozymes are responsible for 20%.

After overnight fast, rats are anesthetized and prepared with a jugular catheter. Dosing group received 9.32 mg/kg tamarixetin (dissolved in DMSO at 10 mg/mL), control group received only DMSO. After 30 minutes, both groups are administered fluvastatin at a dose of 1.5 mg/kg (dissolved in water at 2 mg/mL). Twelve blood samples (including pre dose blank) are collected over 24 hours—0, 10, 20, 40, 60, 120, 240, 360, 480, 720, 1080 and 1440 minutes. Each sample (0.5 to 0.6 mL blood) is collected into microfuge tubes containing 20 uL of 10 IU heparin (anti-coagulant). After separation, plasma samples are protected from light and stored at −80° C. freezer.

Fluvastatin plasma concentration is determined using high performance liquid chromatography with fluorescence detector (excitation 309 nM, emission 390 nm). The LC conditions are, C-18 reverse phase column (5μ, 150×4.6 mm), mobile phase (0.1 M TBAF:0.1M phosphate, pH 6.0:Methanol (15:25:60 v/v/), flow rate 1.0 mL/min, column temperature (50° C.). Analytical procedure is as reported by Toreson et al., (Determination of fluvastatin enantiomers and the racemate in human blood plasma by liquid chromatography and fluorometric detection. Journal of Chromatography A. 729:13-18, 1996).
(1) thaw samples on ice
(2) pippet 250 μL plasma sample into screw cap test tube
(3) add 50 μL of internal standard (celecoxib, 20 μg/mL in MeOH)
(4) add 250 μL of acetonitrile and vortex mixing for 5 seconds
(5) add 250 μL of 0.5 M phosphate buffer, pH 5.0
(6) add 2.5 mL MTBE (methyl tert-butyl ether), shake for 30 minutes
(7) transfer the organic levels into anther test tube, evaporate under reduced pressure
   (8) dissolve extracted residue in mobile phase
(9) transfer the extract and centrifuge at 13000 rpm for 5 minutes
(10) remove the clear supernatant (150 μL) and inject onto HPLC

### Experimental Results

In vitro screening is conducted the essential and adjuvant components of Chinese medicines HUCHE001 to HUCHE070. Inhibition of tolbutamide metabolism in liver microsomes are evaluated at three different concentration range, 1, 10 and 100 μM. For compounds with limited solubility, the highest testing concentration is the highest soluble concentration. The inhibition potential of test compounds is ranked within each testing concentration. The best inhibitors found are: HUCHE070 isoliquritigenin 95.5% inhibition at 100 μM, Tamarixetin 88.2% at 10 μM, HUCHE037 Genistein 49.6% at 1 μM. (Tables 4 to 6).

TABLE 3
introduction of the essential and adjuvant
components of Chinese medicines

| No. | Test article | Name |
|---|---|---|
| HUCHE001 | Genkwanin | Astemisiae Capillaris |
| HUCHE002 | apigenin | Chamomiliae Flos |
| HUCHE003 | luteolin | Digitals Folium |
| HUCHE004 | Luteolin-7-Glucoside | Digitals Folium |
| HUCHE005 | Homoorientin | Swertiae Herba |
| HUCHE006 | sovitexin | Swertiae Herba |
| HUCHE007 | Neohesperidin | Aurantii Fructus Immaturus |
| HUCHE008 | Formononetin | Astragali Radix |
| HUCHE009 | isoliquritigenin | Astragali Radix |
| HUCIIE010 | kaempferol | Sennae Folium |
| HUCHE011 | Isorhamnetin | Sennae Folium |
| HUCHE012 | isoquercitrin | Hydrangeae Dulcis Folium |
| HUCHE013 | (+)-epicatechin | Gambir |
| HUCHE014 | ergosterol | Ergota |
| HUCHE015 | (+)Catechin | Paeoniae Radix |
| HUCHE016 | 6- Gingerol | Zingiberis Rhizoma |
| HUCHE017 | Liquiritin | Glycyrrhizae Radix |
| HUCHE018 | 3-Phenylpropyl Acetate | Cinnamami Cortex |
| HUCHE019 | (−)-Epicatechin | Gambir |
| HUCHE020 | Narigenin | Aurantii Fructus Immaturus |
| HUCHE021 | Umbelliferone | Aurantii Fructus Immaturus |
| HUCHE022 | Rutin | Sophorae Flos |
| HUCHE023 | Hesperidin | Aurantii Fructus Immaturus |
| HUCHE024 | Diosmin | — |
| HUCHE025 | Hesperetin | Citri Reticulatae |
| HUCHE026 | Wongonin | Scutellariae Radix |
| HUCHE027 | baicalin | Scutellariae Radix |
| HUCHE028 | Baicalein | Scutellariae Radix |
| HUCHE029 | Puerarin | Pueraria Radix |
| HUCHE030 | Daidzein | Pueraria Radix |
| HUCHE031 | Daidzin | Pueraria Radix |
| HUCHE032 | Quercitrin | Viscum Coloratum |
| HUCHE033 | quercetin | Viscum Coloratum |
| HUCHE034 | Nordihydroguaiaretic acid | — |
| HUCHE035 | Capillarisin | Artemisia Capillaris |
| HUCHE036 | Swertiamarin | Swertiae Herba |
| HUCHE037 | Genistein | Puerariae Radix |
| HUCHE038 | trans-Cinnamaldehyde | Cinnamami Cortex |
| HUCHE039 | protocatechuic acid | Cinnamami Cortex |
| HUCHE040 | gallic acid | — |
| HUCHE041 | paeoniflorin | Paeoniae Radix |
| HUCHE042 | eriodictyol | Pyracantha Fortuneana |
| HUCHE043 | Poncirin | Aurantii Fructus Immaturus |
| HUCHE044 | α-Naphthoflavone | — |
| HUCHE045 | β-Myrcene | Amomum cardamomum |
| HUCHE046 | α-terpineol | Cinae Flos |
| HUCHE047 | +) -Limonene | Cardamomi Fructus |
| HUCHE048 | Lauryl Alcohol | — |
| HUCHE049 | Ethyl Myristate | Cardamomi Fructus |
| HUCHE050 | Cineole | Cinae Flos |
| HUCHE051 | glycyrrhizin | Glycyrrhizae Radix |
| HUCHE052 | Oleanolic acid | Zizyphi Fructus |
| HUCHE053 | ursolic acid | Zizyphi Fructus |
| HUCHE054 | Narigin | Aurantii Fructus Immaturus |
| HUCHE055 | β-Naphthoflavone | — |
| HUCHE056 | trans-cinnamic acid | Cinnamoni Cortex |
| HUCHE061 | Morin | Mori Radix Cortex |
| HUCHE062 | (+)-Taxifolin | Paeoniae Radix |
| HUCHE063 | Chrysin | Propolis |
| HUCHE064 | Galangin | Zingiberis Rhizoma |
| HUCHE065 | Fisefin | Paeoniae Radix |
| HUCHE066 | myricetin | Hibiscus Abelmoschus |
| HUCHE067 | chrysoeriol | Vernonia Cinerea |
| HUCHE068 | Phloretin | Apple |
| HUCHE069 | Embelin | Ardisia Squamulosa |
| HUCHE070 | Tamarixetin | Tamarix Ramosissima |
| HUCHE071 | sciadopitysin | Ginko Biloba |

TABLE 4
Inhibition of enzymatic activity at 100 μM concentration.

| | | Test article | | % inhi- | |
|---|---|---|---|---|---|
| Code | Test article | conc | | bition | SD |
| — | Ketoconazole | 100 | μM | 100.00 | 0.00 |
| 1 | isoliquritigenin | 100 | μM | 95.47 | 0.15 |
| 2 | Phloretin | 100 | μM | 95.13 | 0.62 |
| 3 | luteolin | 100 | μM | 93.20 | 0.94 |
| 4 | quercetin | 100 | μM | 91.92 | 0.52 |
| 5 | Tamarixetin | 100 | μM | 90.18 | 0.43 |
| 6 | myricetin | 100 | μM | 88.84 | 3.37 |
| 7 | Wongonin | 100 | μM | 84.03 | 2.22 |
| 8 | Genistein | 100 | μM | 82.71 | 2.82 |
| 9 | Nordihydroguaiaretic acid | 100 | μM | 81.18 | 0.50 |
| 10 | Narigenin | 100 | μM | 79.70 | — |
| 11 | Capillarisin | 100 | μM | 79.49 | 3.22 |
| 12 | Chrysin | 50 | μM | 75.11 | 6.05 |
| 13 | Fisefin | 100 | μM | 72.89 | 3.37 |
| 14 | eriodictyol | 100 | μM | 69.62 | 5.68 |
| 15 | 6- Gingerol | 100 | μM | 66.21 | 1.94 |
| 16 | Isorhamnetin | 75 | μM | 65.74 | 4.99 |
| 17 | isoquercitrin | 100 | μM | 61.80 | 15.60 |
| 18 | Formononetin | 50 | μM | 57.94 | 0.84 |
| 19 | Morin | 100 | μM | 51.00 | 4.55 |
| 20 | (+)-Taxifolin | 100 | μM | 50.47 | 10.38 |
| 21 | isovitexin | 100 | μM | 45.36 | 0.97 |
| 22 | 3-Phenylpropyl Acetat | 100 | μM | 42.62 | 2.00 |
| 23 | Oleanolic acid | 100 | μM | 41.13 | 11.52 |
| 24 | ursolic acid | 100 | μM | 38.47 | 3.37 |
| 25 | Puerarin | 100 | μM | 33.30 | 17.52 |
| 26 | β-Myrcene | 100 | μM | 29.85 | 4.31 |
| 27 | trans-cinnamic acid | 100 | μM | 26.10 | 3.57 |
| 28 | Luteolin-7-Glucoside | 100 | μM | 25.08 | 1.57 |
| 29 | Liquiritin | 100 | μM | 24.77 | 8.72 |
| 30 | (+)-Limonene | 100 | μM | 22.29 | 4.04 |
| 31 | Homoorientin | 100 | μM | 20.19 | 11.59 |
| 32 | Swertiamarin | 100 | μM | 18.44 | 2.11 |
| 33 | Embelin | 50 | μM | 17.98 | 4.20 |
| 34 | Daidzein | 25 | μM | 15.74 | 3.24 |
| 35 | Poncirin | 100 | μM | 14.99 | 12.51 |
| 36 | Quercitrin | 100 | μM | 13.48 | 15.69 |
| 37 | (−)Epicatechin | 100 | μM | 5.44 | 4.90 |
| 38 | glycyrrhizin | 100 | μM | 4.87 | 2.73 |
| 39 | ergosterol | 30 | μM | 3.57 | 2.64 |
| 40 | Diosmin | 50 | μM | 3.51 | 1.99 |
| 41 | (+)Catechin | 100 | μM | −0.22 | 6.94 |
| 42 | gallic acid | 100 | μM | −0.97 | 16.40 |
| 43 | Daidzin | 25 | μM | −1.16 | 6.54 |
| 44 | Daidzin | 100 | μM | −1.33 | 7.67 |
| 45 | paeoniflorin | 100 | μM | −1.77 | 3.49 |
| 46 | Umbelliferone | 100 | μM | −2.02 | 5.27 |
| 47 | Rutin | 100 | μM | −6.46 | 13.80 |
| 48 | (+)-epicatechin | 100 | μM | −11.54 | 0.77 |
| 49 | Narigin | 100 | μM | −24.21 | 10.50 |

TABLE 5
Inhibition of enzymatic activity at 10 μM concentration.

| | | Test article | % inhi- | |
|---|---|---|---|---|
| Code | Test article | conc | bition | SD |
| — | Ketoconazole | 10 μM | 80.11 | 0.71 |
| 1 | Tamarixetin | 10 μM | 88.12 | 0.69 |
| 2 | apigenin | 25 μM | 76.88 | 1.37 |
| 3 | Genistein | 10 μM | 67.70 | 2.28 |
| 4 | Isorhamnetin | 10 μM | 61.53 | 3.57 |
| 5 | Chrysin | 10 μM | 60.62 | 2.07 |
| 6 | Wongonin | 10 μM | 51.31 | 1.43 |
| 7 | Narigenin | 10 μM | 49.98 | — |
| 8 | quercetin | 10 μM | 44.80 | 2.37 |
| 9 | Oleanolic acid | 10 μM | 42.35 | 9.56 |
| 10 | Puerarin | 10 μM | 39.02 | 10.00 |
| 11 | kaempferol | 10 μM | 38.29 | 15.43 |
| 12 | luteolin | 10 μM | 37.89 | 14.42 |
| 13 | ursolic acid | 10 μM | 37.46 | 3.31 |
| 14 | isovitexin | 10 μM | 37.38 | 5.79 |
| 15 | Genkwanin | 10 μM | 37.37 | 3.64 |
| 16 | α-Naphthoflavone | 10 μM | 37.27 | 7.06 |
| 17 | Capillarisin | 10 μM | 34.79 | 3.04 |
| 18 | Phloretin | 10 μM | 34.41 | 7.95 |
| 19 | (−)Epicatechin | 10 μM | 33.75 | 13.74 |
| 20 | (+)-Taxifolin | 10 μM | 31.16 | 8.11 |
| 21 | Formononetin | 10 μM | 30.57 | 3.69 |
| 22 | isoliquritigenin | 10 μM | 29.66 | 14.74 |
| 23 | Hesperetin | 10 μM | 29.09 | 2.10 |
| 24 | eriodictyol | 10 μM | 28.65 | 15.29 |
| 25 | 6- Gingerol | 10 μM | 27.72 | 10.54 |
| 26 | isoquercitrin | 10 μM | 27.02 | 17.78 |
| 27 | Fisefin | 10 μM | 26.52 | 7.25 |
| 28 | Quercitrin | 10 μM | 21.10 | 15.81 |
| 29 | Liquiritin | 10 μM | 18.35 | 1.97 |
| 30 | β-Myrcene | 10 μM | 16.60 | 6.31 |
| 31 | Swertiamarin | 10 μM | 16.56 | 3.84 |
| 32 | Poncirin | 10 μM | 16.34 | 10.77 |
| 33 | protocatechuic acid | 10 μM | 16.22 | 1.72 |
| 34 | trans-cinnamic acid | 10 μM | 15.82 | 9.04 |
| 35 | Daidzein | 10 μM | 13.45 | 4.49 |
| 36 | Morin | 10 μM | 11.63 | 17.51 |
| 37 | Embelin | 10 μM | 11.23 | 9.18 |
| 38 | myricetin | 10 μM | 10.57 | 13.21 |
| 39 | (+)-Limonene | 10 μM | 10.55 | 4.18 |
| 40 | Nordihydroguaiaretic acid | 10 μM | 9.76 | 5.26 |
| 41 | ergosterol | 10 μM | 8.12 | 2.19 |
| 42 | baicalin | 25 μM | 7.77 | 3.08 |
| 43 | Hesperidin | 10 μM | 6.68 | 3.32 |
| 44 | (+)-epicatechin | 10 μM | 6.30 | 3.72 |
| 45 | Baicalein | 25 μM | 5.06 | 8.64 |
| 46 | Diosmin | 10 μM | 4.70 | 0.75 |
| 47 | β-Naphthoflavone | 10 μM | 4.64 | 3.02 |
| 48 | Homoorientin | 10 μM | 2.45 | 13.94 |
| 49 | glycyrrhizin | 10 μM | 2.23 | 4.65 |
| 50 | paeoniflorin | 10 μM | 0.70 | 3.50 |
| 51 | Luteolin-7-Glucoside | 10 μM | −0.32 | 5.20 |
| 52 | Daidzin | 10 μM | −2.46 | 4.10 |
| 53 | gallic acid | 10 μM | −2.47 | 10.16 |
| 54 | Umbelliferone | 10 μM | −6.64 | 4.94 |
| 55 | (+)Catechin | 10 μM | −8.46 | 3.53 |
| 56 | (−)-Epicatechin | 10 μM | −8.61 | 5.95 |
| 57 | Narigin | 10 μM | −13.25 | 4.33 |
| 58 | Rutin | 10 μM | −13.97 | 14.31 |

TABLE 6
Inhibition of enzymatic activity at 1 μM

| | | Test article | % inhi- | |
|---|---|---|---|---|
| Code | Test article | conc | bition | SD |
| — | Ketoconazole | 1 μM | 45.88 | 3.13 |
| 1 | Genistein | 1 μM | 49.60 | 1.37 |
| 2 | Tamarixetin | 1 μM | 41.96 | 6.63 |
| 3 | Puerarin | 1 μM | 38.15 | 0.57 |
| 4 | 3-Phenylpropyl Acetate | 1 μM | 36.57 | 7.30 |
| 5 | isovitexin | 1 μM | 35.56 | 7.96 |
| 6 | ursolic acid | 1 μM | 33.62 | 0.99 |
| 7 | eriodictyo | 1 μM | 32.78 | 4.41 |
| 8 | Genkwanin | 1 μM | 30.85 | 1.68 |
| 9 | 6- Gingerol | 1 μM | 30.17 | 2.36 |
| 10 | Wongonin | 1 μM | 28.82 | 1.41 |
| 11 | trans-cinnamic acid | 1 μM | 26.92 | 4.26 |
| 12 | Embelin | 1 μM | 24.71 | 6.18 |
| 13 | Quercitrin | 1 μM | 24.19 | 1.71 |
| 14 | β-Myrcene | 1 μM | 24.06 | 3.08 |
| 15 | Phloretin | 1 μM | 23.76 | 6.21 |
| 16 | Formononetin | 1 μM | 23.33 | 0.43 |
| 17 | apigenin | 2.5 μM | 21.69 | 1.37 |
| 18 | isoquercitrin | 1 μM | 20.94 | 1.96 |
| 19 | protocatechuic acid | 1 μM | 20.26 | 9.00 |
| 20 | luteolin | 1 μM | 20.09 | 21.27 |
| 21 | Isorhamnetin | 1 μM | 19.63 | 6.32 |
| 22 | Capillarisin | 1 μM | 19.33 | 7.81 |
| 23 | Liquiritin | 1 μM | 18.10 | 9.70 |
| 24 | (+)-epicatechin | 1 μM | 16.99 | 2.53 |
| 25 | Oleanolic acid | 1 μM | 16.79 | 1.67 |
| 26 | Swertiamarin | 1 μM | 16.33 | 0.92 |
| 27 | quercetin | 1 μM | 15.11 | 1.03 |
| 28 | Morin | 1 μM | 14.26 | 2.86 |
| 29 | (+)-Limonene | 1 μM | 14.12 | 3.63 |
| 30 | paeoniflorin | 1 μM | 10.11 | 4.34 |
| 31 | Luteolin-7-Glucoside | 1 μM | 9.37 | 3.17 |
| 32 | Poncirin | 1 μM | 7.76 | 6.36 |
| 33 | Chrysin | 1 μM | 6.86 | 2.17 |
| 34 | Fisefin | 1 μM | 5.49 | 7.50 |
| 35 | Narigenin | 1 μM | 5.20 | — |
| 36 | glycyrrhizin | 1 μM | 5.14 | 6.63 |
| 37 | Homoorientin | 1 μM | 3.37 | 8.22 |
| 38 | Hesperidin | 1 μM | 2.57 | 2.07 |
| 39 | β-Naphthoflavone | 1 μM | 2.35 | 4.87 |
| 40 | Baicalein | 2.5 μM | 1.76 | 2.53 |
| 41 | Diosmin | 1 μM | 1.51 | 0.82 |
| 42 | Daidzein | 1 μM | 1.35 | 1.54 |
| 43 | (−)-Epicatechin | 1 μM | 1.11 | 4.15 |
| 44 | ergosterol | 1 μM | 1.00 | 0.59 |
| 45 | Daidzin | 1 μM | 0.95 | 3.51 |
| 46 | isoliquritigenin | 1 μM | 0.87 | 5.00 |
| 47 | α-Naphthoflavone | 1 μM | −0.05 | 6.26 |
| 48 | (+)-Taxifolin | 1 μM | −1.29 | 8.16 |
| 49 | Rutin | 1 μM | −2.59 | 12.71 |
| 50 | gallic acid | 1 μM | −3.05 | 5.18 |
| 51 | (+)Catechin | 1 μM | −3.05 | 0.78 |
| 52 | myricetin | 1 μM | −3.19 | 16.64 |
| 53 | Hesperetin | 1 μM | −3.58 | 11.11 |
| 54 | baicalin | 2.5 μM | −5.36 | 6.97 |
| 55 | Umbelliferone | 1 μM | −7.17 | 3.59 |
| 56 | Narigin | 1 μM | −11.48 | 2.10 |
| 57 | Nordihydroguaiaretic acid | I μM | −16.06 | 2.77 |
| 58 | kaempferol | 1 μM | −22.27 | 18.96 |

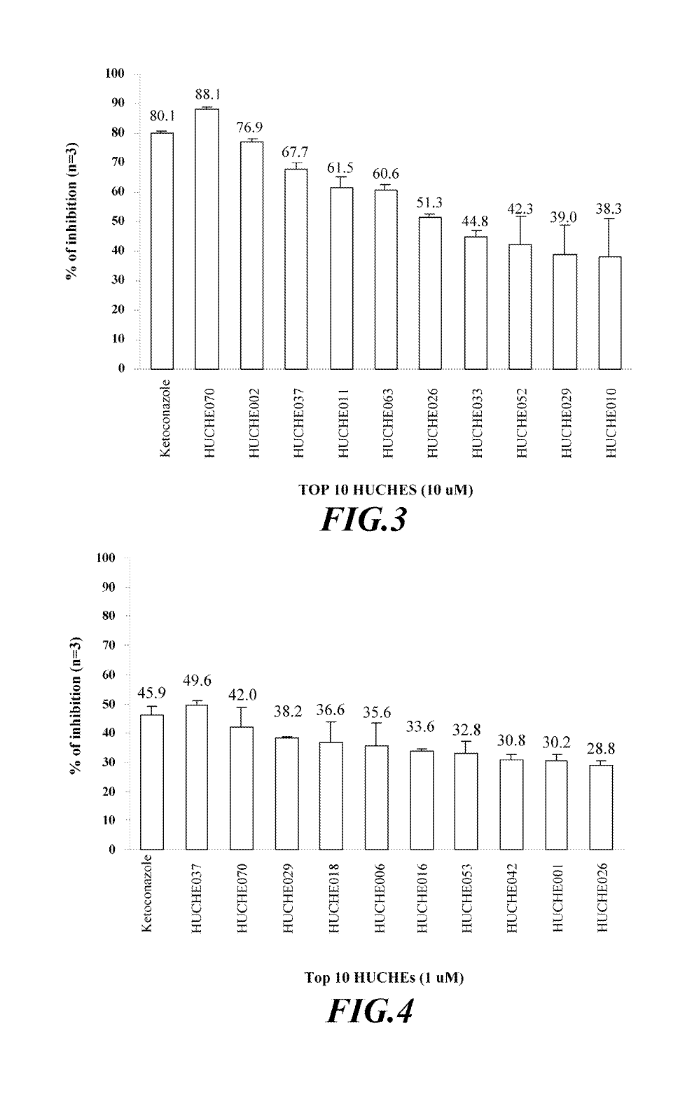

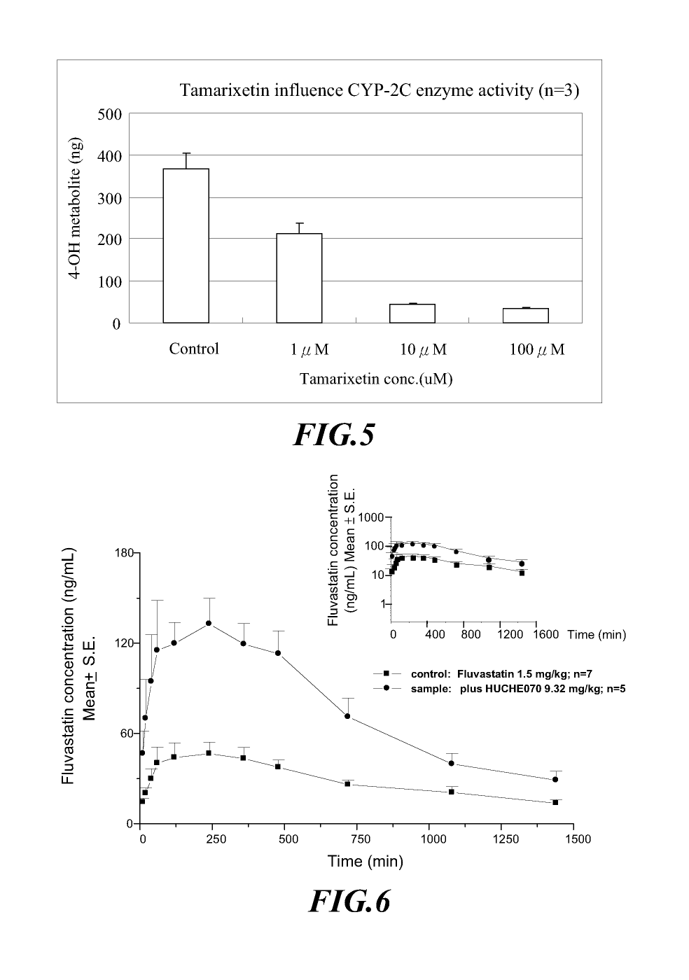

Student T-test is performed on the inhibition data to assess the statistical significance of observed effects relative to the control group. Results from the best 10 test compounds at 100, 10 or 1 μM concentration are depicted in FIGS. 2 to 4.

### Specific Example 1

Using the procedure described in previous section, the inhibitory effect of HUCHE070 Tamarixetin against the microsomal metabolism of tolbutamide is evaluated at different concentrations. The reaction conditions are: tolbutamide 1 mM, microsomal protein 0.5 mg, reaction time 7.5 minute. Test results indicated HUCHE070 Tamarixetin is an inhibitor. The % inhibition is 90.2, 88.1 and 42.0% at the high, mid and low concentration, respectively. It is concluded that Tamarixetin is an effective CYP2C9 inhibitor.

TABLE 7
In vitro effects of HUCHE070 on the metabolism
of tolbutamide in microsomes (n = 3)

| Concentration | 4′-hydroxytolbutamide (ng) | % inhibition |
|---|---|---|
| Control | 368.5409 ± 35.3091 | 0.0000 |

| | | | |
|---|---|---|---|
| 1 | μM | 213.5696 ± 24.4309 | 41.9620 |
| 10 | Mm | 43.10052 ± 2.5372 | 88.1204 |
| 100 | μM | 35.49297 ± 1.5825 | 90.1803 |

Effects of Tamarixetin on oral bioavailability of fluvastatin in Sprague Dawley rats are summarized in Tables 8 and 9. Pharmacokinetic parameters obtained for both treatment groups are presented in Table 10. Plasma fluvastatin concentration vents time curves are depicted in FIG. 6. Pharmacokinetic analysis indicated that there are differences in the AUC (area under the plasma concentration time curve) values. The Cmax for the treatment group is 141.4±15.8 ng/mL, two-fold higher than the value (63.1±10.4 ng/mL) for the control group. Estimates of plasma clearance (CL) and volume of distribution (Vd) are also different between the treatment and the control groups, suggesting inhibition of hepatic metabolism. There is no apparent changes of terminal elimination rate constant (k), and therefore the half-life (T_{1/2}) of both groups are not different. These results indicated that Tamarixetin did not exhibit a persisted inhibition of the metabolic activity, and fluvastatin is eliminated and excreted from the animal body by the regular pathways.

TABLE 8
Blood concentration of fluvastatin in Sprague-Dawley rats
following oral administration in the control group.

| Time | Concentration (ng/ml) | | | | | | | | | |
|---|---|---|---|---|---|---|---|---|---|---|
| (min) | C-1 | C-2 | C-3 | C-4 | C-5 | C-6 | C-7 | mean | SE | CV % |
| 10 | 13.56 | 16.39 | 19.78 | 24.70 | 0.27 | 8.94 | 16.41 | 14.3 | 3.0 | 55.2 |
| 20 | 28.95 | 18.03 | 18.10 | 24.48 | 1.15 | 19.79 | 31.50 | 20.3 | 3.8 | 49.1 |
| 40 | 47.38 | 22.74 | 25.67 | 29.33 | 7.35 | 63.98 | 42.36 | 34.1 | 7.0 | 54.5 |
| 60 | 69.48 | 24.74 | 34.71 | 41.24 | 7.57 | 99.41 | 44.17 | 45.9 | 11.4 | 65.9 |
| 120 | 61.69 | 29.13 | 43.91 | 54.63 | 11.19 | 98.41 | 41.14 | 48.6 | 10.4 | 56.6 |
| 240 | 54.64 | 38.66 | 66.97 | 73.42 | 14.48 | 68.72 | 38.28 | 50.7 | 8.1 | 42.1 |
| 360 | 40.68 | 45.74 | 60.75 | 83.84 | 20.41 | 50.80 | 27.07 | 47.0 | 8.0 | 45.2 |
| 480 | 37.37 | 57.30 | 45.05 | 54.92 | 21.57 | 37.68 | 24.92 | 39.8 | 5.2 | 34.4 |
| 720 | 22.45 | 37.37 | 25.63 | 39.30 | 21.75 | 18.47 | 15.40 | 25.8 | 3.5 | 35.6 |
| 1080 | 16.11 | 35.39 | 22.58 | 36.80 | 14.10 | 10.67 | 11.68 | 21.1 | 4.2 | 52.6 |
| 1440 | 11.47 | 22.33 | 21.04 | — | 10.74 | 6.33 | 8.58 | 13.4 | 2.7 | 51.5 |

TABLE 9
Blood concentration of fluvastatin and HUCHE070 Tamarixetin in Sprague-
Dawley rats following oral administration in the test group.

| Time | Concentration (ng/ml) | | | | | | | |
|---|---|---|---|---|---|---|---|---|
| (min) | S-1 | S-2 | S-3 | S-4 | S-5 | mean | SE | CV % |
| 10 | 59.54 | 18.79 | 29.52 | 100.09 | 25.45 | 46.68 | 15.07 | 72.2 |
| 20 | 137.55 | 16.78 | 35.58 | 127.52 | 32.38 | 69.96 | 25.79 | 82.4 |
| 40 | 186.33 | 38.82 | 45.28 | 153.00 | 49.34 | 94.55 | 31.16 | 73.7 |
| 60 | 190.51 | 45.28 | — | 150.29 | 74.50 | 115.15 | 33.48 | 58.1 |
| 120 | 155.29 | 107.75 | 83.64 | 150.05 | 102.20 | 119.78 | 14.03 | 26.2 |
| 240 | 110.95 | 110.19 | 160.57 | 185.52 | 97.33 | 132.91 | 17.02 | 28.6 |
| 360 | 88.95 | 97.35 | 146.50 | 157.95 | 106.03 | 119.36 | 13.81 | 25.9 |
| 480 | 71.17 | 86.62 | 116.44 | 153.96 | 136.59 | 112.95 | 15.32 | 30.3 |
| 720 | 55.50 | 60.50 | 97.19 | 103.45 | 38.63 | 71.05 | 12.53 | 39.4 |
| 1080 | 28.45 | — | — | 50.99 | — | 39.72 | 11.27 | 40.1 |
| 1440 | 27.61 | — | — | 41.51 | 18.00 | 29.04 | 6.82 | 40.7 |

TABLE 10
Pharmacokinetics of fluvastatin in Sprague-
Dawley rats following oral administration.

| | | Fluvastatin(B) control, | Fluvastatin (A) with | |
|---|---|---|---|---|
| PK parameter | (unit) | n = 7 | Tamarixetin , n = 5 | A/B |
| Cₘₐₓ | (ng/mL) | 63.14 ± 10.36 | 141.40 ± 15.76* | 2.4 |
| Tₘₐₓ | (hr) | 4.7 ± 1.7 | 4.2 ± 1.1 | 0.9 |
| AUCₜ | (hr*ng/mL) | 710.57 ± 81.55 | 1389.20 ± 166.14* | 2.0 |
| AUC_{INF} | (hr*ng/mL) | 949.86 ± 133.48 | 2281.00 ± 386.56* | 2.4 |
| K | (1/hr) | 0.074 ± 0.005 | 0.065 ± 0.009 | 0.9 |
| T_{1/2} | (hr) | 9.7 ± 0.65 | 11.3 ± 1.33 | 1.2 |
| Cl/F | (mL/min/kg) | 29.12 ± 4.05 | 12.33 ± 2.10 ** | 0.4 |
| Vz/F | (mL/kg) | 24846.64 ± 4721.23 | 11163.54 ± 861.69* | 0.4 |
| AUMC_{INF} | (hr*hr*ng/mL) | 15156.0 ± 2864.6 | 42896.4 ± 12379.8 | 2.8 |
| MRT_{INF} | (hr) | 15.82 ± 1.56 | 17.31 ± 2.27 | 1.1 |

PK = pharmacokinetic,
Data = mean ± SE,
*p < 0.05,
** P < 0.01

### Specific Example 2

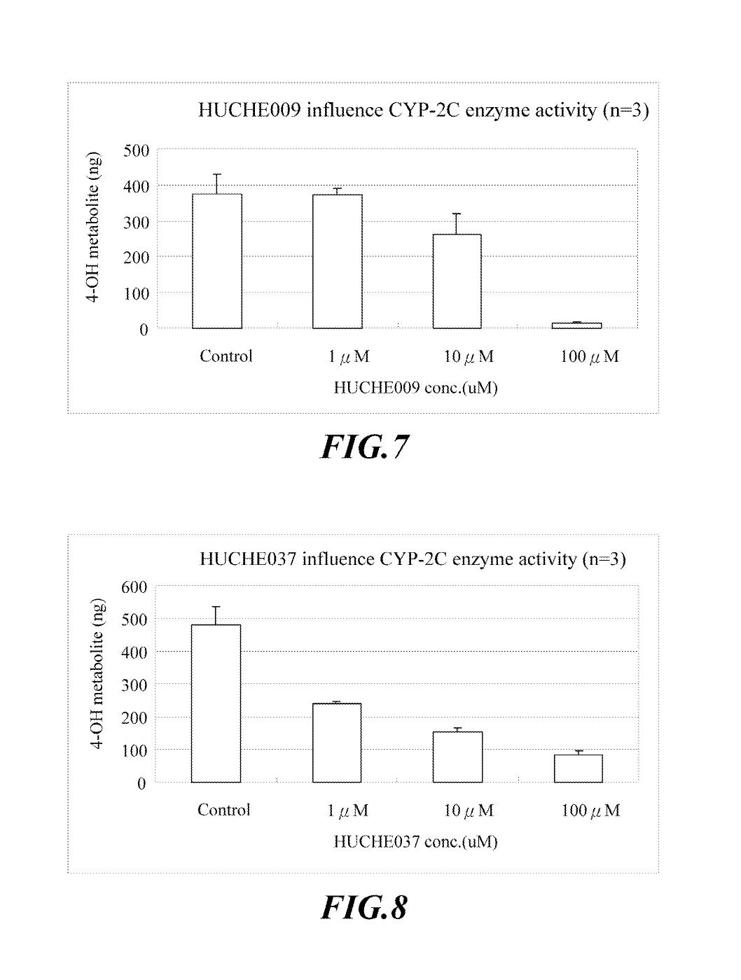

Using the procedure described in previous section, the inhibitory effect of HUCHE009 isoliquritigenin against the microsomal metabolism of tolbutamide is evaluated at different concentrations. The reaction conditions are: tolbutamide 1 mM, microsomal protein 0.5 mg, reaction time 7.5 minute. Test results (Table 11 and FIG. 7) indicated HUCHE009 isoliquritigenin inhibited 95.46% of the activity at the high concentration. It is considered that HUCHE009 isoliquritigenin is an effective CYP2C9 inhibitor.

TABLE 11
In vitro effects of HUCHE009 isoliquritigenin on the
metabolism of tolbutamide in microsomes (n = 3)

| Concentration | 4′-hydroxytolbutamide (ng) | % inhibition |
|---|---|---|
| Control | 374.8785 ± 54.8521 | 0.0000 |

| | | | |
|---|---|---|---|
| 1 | μM | 371.5965 ± 18.7272 | 0.8737 |
| 10 | Mm | 263.4592 ± 55.2455 | 29.6603 |
| 100 | μM | 16.25213 ± 0.5544 | 95.4680 |

### Specific Example 3

Using the procedure described in previous section, the inhibitory effect of HUCHE037 Genistein against the microsomal metabolism of tolbutamide is evaluated at different concentrations. The reaction conditions are: tolbutamide 1 mM, microsomal protein 0.5 mg, reaction time 7.5 minute. Test results indicated HUCHE037 Genistein is an inhibitor. The % inhibition is 82.7, 67.7 and 49; 6% at the high, mid and low concentration, respectively. It is concluded that HUCHE037 Genistein is an effective CYP2C9 inhibitor.

TABLE 12
n vitro effects of HUCHE037 Genistein on the metabolism
of tolbutamide in microsomes (n = 3)

| Concentration | 4′-hydroxytolbutamide (ng) | % inhibition |
|---|---|---|
| Control | 479.3314 ± 56.4829 | 0.0000 |

| | | | |
|---|---|---|---|
| 1 | μM | 241.2098 ± 6.5885 | 49.5979 |
| 10 | Mm | 154.311 ± 10.9480 | 67.6979 |
| 100 | μM | 82.24342 ± 13.3679 | 82.7088 |

What is claim is:

1. A pharmaceutical combination for enhancing the bioavailability of a therapeutic agent, comprising:
a pharmaceutically effective CYP2C9 inhibitor with concentration ranged from 1 μM to 100 μM and said pharmaceutically effective CYP2C9 inhibitor is Tamarixetin; and
a pharmaceutically viable drug extensively metabolized by CYP2C9;
wherein said pharmaceutically viable drug is fluvastatin.
